(19) United States
(12) Patent Application Publication
(10) Pub. No.: US 2016/0349271 A1
(43) Pub. Date: Dec. 1, 2016

(54) BIOMARKERS FOR CARDIOVASCULAR DISEASE

(71) Applicant: DIADEXUS, INC., South San Francisco, CA (US)

(72) Inventors: Shaoqiu ZHUO, Moraga, CA (US); Maria Teresa JALILIE, San Bruno (US)

(21) Appl. No.: 15/117,056

(22) PCT Filed: Feb. 13, 2015
(86) PCT No.: PCT/US15/15952

§ 371 Date: Aug. 5, 2016

Related U.S. Application Data
(60) Provisional application No. 61/940,200, filed on Feb. 14, 2014.
(60) Provisional application No. 62/065,576, filed on Oct. 17, 2014.

(51) Int. Cl. G01N 33/68

(52) U.S. Cl. CPC G01N 33/6893; G01N 2333/918; G01N 2800/324; G01N 2800/32; G01N 2800/52; G01N 2800/56

(57) ABSTRACT

Described herein are methods for diagnosing or assessing and treating an individual for cardiovascular disease based on the individual's normalized level of biomarkers. For example, a level of Lp-PLA₂ mass or Lp-PLA₂ activity normalized to a level of Lp-PLA₂ total mass (e.g. total mass) may be used. Described herein are new and more accurate diagnostic indicators to help identify and stratify individuals having cardiovascular disease or at risk for cardiovascular disease, as well as methods for treating such patients. The methods and techniques described herein may be especially useful for detecting early stages of cardiovascular disease, and may be particularly useful for distinguishing a person having cardiovascular disease from a person without cardiovascular disease.

### CROSS REFERENCE TO RELATED APPLICATIONS

This patent application claims priority to U.S. provisional patent application No. 61/940,200; filed 14 Feb. 2014 (“BIOMARKERS FOR CARDIOVASCULAR DISEASE”) and U.S. provisional patent application No. 62/065,576; filed 17 Oct. 2014 (“BIOMARKERS FOR CARDIOVASCULAR DISEASE”).

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are compositions, kits, and methods using biomarkers for identifying cardiovascular disease, including diagnosing and prognosticating cardiovascular disease, and for treating cardiovascular disease.

### BACKGROUND

Cardiovascular disease (CVD)—including heart disease and coronary artery disease—is the leading cause of death in the United States. About 600,000 people die of heart disease each year and many more suffer from pain and a diminished lifestyle due to cardiovascular disease. Early detection of cardiovascular disease and prediction of future risk of cardiovascular disease are key factors to reducing or even preventing progression of cardiovascular disease. Although some risk factors for cardiovascular disease have been described, it remains a significant, costly and unsolved problem.

CVD is not the result of one single disease state, but, rather it is a complex syndrome spanning a broad range of pathophysiological features including myocyte injury/stress, inflammation/oxidative stress, neurohormonal responses to decompensation, extracellular matrix remodeling, and renal dysfunction. Cardiovascular disease may be diagnosed by identifying abnormal or altered features. Heart failure, for example, is currently determined using a variety of tests to place the degree of heart failure into one of four classes from I to IV using the New York Heart Association (NYHA) Functional Classification system. In this system, Class I heart failure is the least severe, with no symptoms of heart failure and class IV is the most severe. Tests used for classifying heart failure may include analysis of a blood sample that is assayed for increased levels of B-type natriuretic peptide (BNP) which is indicative of heart failure. Early and appropriate intervention of cardiovascular disease leads to the best outcomes. Although CVD is common, its diagnosis is often missed. It may be missed, for example, because a person may have no symptoms (e.g. such as a person with NYHA Class I heart failure) and therefore does not get tested, or a person may be mis-diagnosed with a different disease that has similar symptoms, or might not be tested because a test is dangerous, expensive, unavailable, or gives ambiguous or false results.

Existing diagnostic tests may be problematic. For example, BNP/pro-BNP tests may be less reliable in obese patients or patients with renal failure. Thus, there is a need for more reliable assays and treatment methods, as well as more effective markers to identify and stratify individuals having cardiovascular disease.

LpPLA₂ has been previously proposed as a biomarker for use in predicting outcomes for patients diagnosed with heart failure and only for patients within NYHA class III and IV. See, e.g., Gerber, Y., et al. Plasma lipoprotein-associated phospholipase A2 levels in heart failure: Association with mortality in the community; Atherosclerosis 203 (2009) 593-598; Van Vark, L. C., et al. Lipoprotein-associated phospholipase A2 activity and risk of heart failure: the Rotterdam Study. European Heart Journal (2006) 27, 2346-2352; and Schott and Berg, Medical Affairs Bulletin; Biomarkers in Heart Failure: Lp-PLA₂ (activity) was predictive of incident heart failure in an at-risk population and was prognostic in a population with heart failure (Lp-PLA₂ mass). For example, the Gerber et al. paper specifically references only NYHA class >3, and even then shows only a dubious statistical significance (p=0.26; See Table 1 of Gerber). While Lp-PLA2 levels have been useful for diagnosing some stages of CVD, there is room for increased specificity and sensitivity both in better diagnosing CVD that may previously have been missed and for reclassifying individuals classified as symptom free as having cardiovascular disease.

Described herein are techniques that may be particularly useful for diagnosing or assessing CVD. Specifically, the techniques described herein may be used to diagnose or reclassify individuals with cardiovascular disease.

Although both the detection of the amount (e.g., mass) of LpPLA2 in a patient sample, as well as the detection of Lp-PLA2 activity from a patient sample have been looked at previously, surprisingly the two assays do not appear to provide correlated information. As illustrated in FIG. 21, illustrating the results of both mass and activity assays performed on the same patients, there is no correlation between the two assays.

For example, described herein are techniques for assaying Lp-PLA₂ that differ from existing assays. Also described are techniques that use a first detectable level of the biomarker Lp-PLA₂ (also referred to as PLAC or PAF-AH) in combination with a second detectable level of Lp-PLA₂ that is different from the first detectable level to identify cardiovascular disease patients from apparently healthy donors without cardiovascular disease, to reclassify individuals with cardiovascular disease, and to treat the CVD patients for cardiovascular disease.

### SUMMARY OF THE DISCLOSURE

Described herein are new and more accurate diagnostic indicators to help identify and stratify individuals having cardiovascular disease or at risk for cardiovascular disease, as well as methods for treating such patients. The methods and techniques described herein may be especially useful for detecting early stages of cardiovascular disease, and may be particularly useful for distinguishing a person having cardiovascular disease from a person without cardiovascular disease. The methods and techniques described herein may be especially useful for distinguishing one or more than one different types of cardiovascular disease, including, but not limited to acute myocardial infarction, hemorrhagic stroke, ischemic heart disease (IHD)/hypertension, ischemic stroke, other cerebrovascular diseases, and peripheral artery disease. For example, described herein are biomarkers that may be used alone or used in combination for diagnosing and treating cardiovascular disease. The disclosure also provides methods for preventing further cardiovascular disease, treating an existing case of cardiovascular disease, or ameliorating the effects from cardiovascular disease. These methods may be based on, for example, the diagnosis or prognosis of cardiovascular disease by one or more biomarkers.

A biomarker (which is short for “biological marker”) may be a characteristic that is objectively measured and evaluated as an indicator of a normal biological process, a pathogenic process, or a pharmacologic response to an intervention. For example, a biomarker may include Lp-PLA₂ (standard mass), Lp-PLA₂ (total mass), and Lp-PLA₂ (activity), Lp(a), TGLIP, apoA1, total cholesterol, LDL-cholesterol, or HDL-cholesterol.

In some examples, a single biomarker may be used to perform the methods described herein. In other cases, a combination of CVD biomarkers may be chosen. The biomarkers may represent one or more than one pathophysiologic category, such as myocyte injury/stress, inflammation/oxidative stress, neurohormonal responses to decompensation, extracellular matrix remodeling, and renal dysfunction, and may be beneficial, especially if the combination may provide more accurate diagnostic, prognostic, prevention or treatment information regarding the earliest stages of cardiovascular disease relative to a healthy patient population. Such orthogonal markers, e.g., markers for two different pathophysiologic categories of a disease syndrome may be utilized to diagnose or prognosticate cardiovascular disease. As described herein using Lp-PLA₂ standard mass or Lp-PLA₂ activity in combination with an Lp-PLA₂ total mass improved diagnostic and prognostic capability for cardiovascular disease. Lp-PLA₂ standard mass, as currently measured for assessing cardiovascular disease, is a marker of inflammation/oxidative stress. However, only a portion of Lp-PLA₂ mass in the blood is currently measured and the exact role that Lp-PLA₂ might play in inflammation and disease progression is not clear. For example, using a new mass assay (“total” or “modified mass” assay) that detects significantly more (or essentially all) Lp-PLA₂ in a blood sample, we show that the existing Lp-PLA₂ standard mass assay for diagnosing CVD detects only about 10%-50% of the Lp-PLA₂ mass in the blood. In particular, the assay may preferentially detect Lp-PLA₂ associated with HDL and not Lp-PLA₂ associated with LDL and VLDL. Such as assay may minimize “noise” associated with an assay and provide more consistent results. Analyzing the measured analyte values by nominal logistic regression, we demonstrate here that the level of the specific biomarker, Lp-PLA₂^{Mass} as assayed as a marker for inflammation/oxidative stress in combination with a more complete assay detecting previously undetected Lp-PLA2mass and especially the normalized value (ratio) of the level of Lp-PLA₂^{standard mass} to the level to Lp-PLA2^{total mass} together provide diagnostic or prognostic value for cardiovascular disease. We also demonstrate that the level of the specific biomarker, Lp-PLA₂^{Activity} combination with the more complete assay detecting previously undetected Lp-PLA₂ mass and especially the normalized value (ratio) of the level of Lp-PLA₂^{Activity} to the level to Lp-PLA₂^{total mass} together provide diagnostic or prognostic value for cardiovascular disease. A normalized value is the ratio of the total amount of Lp-PLA₂^{standard mass} or Lp-PLA₂^{Activity} detected to the total amount of Lp-PLA2 standard mass (using an Lp-PLA₂^{total mass} assay, regardless of whether they are associated together (e.g. whether are on the same particle in the blood).

Utilizing a cohort of one hundred and forty one samples comprising eighty-five donor samples from patients having cardiovascular disease (including twenty-nine ischemic and thirteen hemorrhagic stroke, and twenty-five acute myocardial infarction, seventeen ischemic heart disease) and fifty-six apparently healthy donor samples, the levels of individual analytes were measured and the ratios (or normalized values) of two biomarkers together were analyzed. In one example, Lp-PLA₂^{standard mass} and Lp-PLA₂^{total mass} were measured and the level of Lp-PLA₂^{mass} was normalized to the level of Lp-PLA₂^{total mass}. Analyzing the measured analyte values by ordinal logistic regression, we additionally demonstrate here that the combination of these two analytes (Lp-PLA₂^{standard mass} and Lp-PLA₂^{total mass}) provided excellent specificity and sensitivity (i.e., ROC curves) for discriminating individuals with cardiovascular disease from the apparently healthy donors. In another example, Lp-PLA₂^{Activity} and Lp-PLA₂^{total mass} were measured and the level of Lp-PLA₂^{Activity/}Lp-PLA₂^{total mass} was determined. Analyzing the measured analyte values by ordinal logistic regression, we additionally demonstrate here that the combination of these two analytes (Lp-PLA₂^{Activity} and Lp-PLA₂^{total mass}) provided excellent specificity and sensitivity (i.e., ROC curves) for discriminating individuals with cardiovascular disease from the healthy donors. Such discrimination may include broadly discriminating cardiovascular disease or may include discriminating a subclass of cardiovascular disease, such as ischemic heart disease, acute myocardial infarction, hemorrhagic stroke, or ischemic stroke. In general, any combination of Lp-PLA2 activity and Lp-PLA2 mass may be expressed, and is not limited to (though includes) a simple percentage of mass/activity or activity/mass. For example, a combination of Lp-PLA₂^{Activity} and Lp-PLA₂^{total mass} (or Lp-PLA₂^{Activity} and Lp-PLA₂^{mass}) may be based on the relationship between patients above a risk threshold for a disease (e.g. coronary disease). Examples of such combinations may linear and non-linear relationships between activity and mass (either with or without detergent) that may provide an estimate of risk.

The results demonstrate a clinical threshold for use of biomarkers in cardiovascular disease diagnosis, reclassification or prediction. A threshold may be a cut-point based on measured values (e.g. a Youden or J value based on sensitivity and specificity) or may otherwise be chosen to provide any percent of disease detection, such as greater than 50%, 60%, 70%, 80%, 90%, 91%, 92%, 93%, 94%, 95%, 96%, 97%, 98%, or 99% of disease detection or may be a value that is between any two of these values. A value between these values may, for example, correspond to a value that is read from a graph or based on a statistical analysis that falls between two of the above listed categories. For example, a cut-point or other threshold value may be chosen to provide higher specificity or to provide higher sensitivity. A particular cut-point or other threshold value may be chosen so as to be utilized along with another biomarker(s) (including any ratios or normalization levels of biomarker) including any of those described herein for assaying disease or risk of disease which together may improve specificity or sensitivity. A cut-point or other value may be chosen to be utilized along with another factor such as a risk factor (e.g. smoking status) which together may improve specificity or sensitivity.

Additionally, a threshold for a biomarker that may be used alone or along with a ratio or normalization level for two biomarkers may be chosen to provide any level of CVD detection. A threshold range for normalized values may be greater than 0.20, 0.21, 0.22, 0.23, 0.24, 0.25, 0.26, 0.27, 0.28, 0.29, 0.30, 0.35, or 0.40.

For example, a threshold level of Lp-PLA₂ may be greater than 200 ng/ml, 300 ng/ml, 400 ng/ml, 500 ng/ml, 600 ng/ml, 700 ng/ml, 800 ng/ml, 1000 ng/ml, 1200 ng/ml, or 1400 ng/ml or a value between any two of these values in a blood (plasma or serum) sample. For example, a threshold of Lp-PLA₂ activity may be greater than 150 nmol/min/ml, greater than 160 mol/min/ml, greater than 170 mol/min/ml, greater than 180 mol/min/ml, greater than 190 mol/min/ml, or greater than 200 mol/min/ml. A value between these values may correspond to a value that is read from a graph or based on statistical analysis that falls between two of the above listed categories. A range may have an upper threshold of less than 300 ng/ml, 400 ng/ml, 500 ng/ml, 600 ng/ml, 700 ng/ml, 800 ng/ml, 1000 ng/ml, 1200 ng/ml, or 1400 ng/ml. The results also demonstrate that a range of values may be useful. A range may have both a lower threshold and a threshold as listed above. For example, a particular upper threshold or a lower threshold may be chosen depending on which other factors are being considered for a diagnosis or prognostication (e.g., risk) (e.g., other test results, other diagnoses, patient symptoms, family history, etc.). Minimal and maximum threshold values may be chosen to assign a diagnosis or risk level; for example to place a sample into a one particular subclass from a range of multiple subclasses.

Minimal and maximum values of a cut-point may be chosen to assign a diagnosis or risk level; for example to place a sample into a one particular subclass from a range of multiple subclasses.

A treatment for cardiovascular disease may include, for example, aldosterone blockers, angiotensin-converting enzyme (ACE) inhibitors, angiotensin-receptor blockers (ARBs), aspirin, beta blockers, diuretics, digitalis, hydralazine and nitrates, statins, and warfarin.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a comparison of sizes and densities for various types of submicroscopic lipoprotein particles, which contain a protein molecule wrapped around cholesterols and fats to transport cholesterol and fats through the blood for use by various tissues in the body. FIG. 1 also points to a subset of lipoprotein particles that form unwanted plaque deposits on the insides of blood vessels and are implicated in heart and artery diseases.

FIG. 2A shows the general structure of a lipoprotein shown in FIG. 1, called apoB-100 lipoproteins, which have a single particle of an apoliproteinB-100 protein wrapped around cholesterols and fats and that transport the cholesterol and fats through the blood and into cells.

FIG. 2B shows the general structure of one type of lipoprotein such as shown in FIG. 2A, called lipoprotein (a) (Lp(a)), with a particle of apolipoprotein(a) protein attached to the particle of apoliproteinB-100 protein. High levels of Lp(a) in the bloodstream have been implicated in heart and artery disease.

FIG. 2C shows a space filling model of high-density lipoprotein (HDL).

FIG. 2D shows the steps of forming a high-density lipoprotein (HDL) particle, such as one shown in FIG. 1, by adding additional molecules to the particle.

FIG. 3 shows an overview of the molecules involved with lipid and cholesterol synthesis and transport in the blood and how organs in the body form and change some of these molecules, especially lipoprotein particles.

FIG. 4A shows a hypothetical graphical example of a statistically significant result for a biomarker that would be useful for detecting cardiovascular disease using receiver operating characteristic (ROC) analysis.

FIG. 4B shows a description of the accuracy of statistical analyses of a ROC curve such as the one shown in FIG. 4A.

FIG. 5 shows the characteristics of a cohort of patients with cardiovascular disease and a control group population without cardiovascular disease tested for biomarkers for cardiovascular disease as described herein.

FIGS. 6A-C show histograms of data distribution of results from blood samples taken from the individuals in the cohorts shown in FIG. 5 and tested for levels of biomarkers Lp-PLA₂ mass, Lp-PLA2 total mass (total mass assay), and Lp-PLA₂ activity of cardiovascular disease. FIGS. 6A-C also show basic statistical analysis of the results, including mean values, median values, quartiles, and standard deviations.

FIGS. 7A-C show a statistical one-way analysis of variance (ANOVA) of the results shown in FIGS. 6A-L, for determining if levels of the candidate biomarker Lp-PLA₂ which are analyzed in different ways (standard mass, total mass (in CHAPS), and activity) vary based on the type of blood sample (blood plasma vs blood serum) used for testing.

FIGS. 8A-C show a statistical one-way analysis of variance (ANOVA) of the results shown in FIGS. 6A-C for determining if the levels of the candidate biomarker Lp-PLA₂ analyzed in different ways vary based on gender (male vs female).

FIGS. 9A-C show a statistical bivariate analysis of the results shown in FIGS. 6A-L for determining if the levels of the biomarker Lp-PLA₂ analyzed in different ways vary based on the patient's age.

FIGS. 10A-C show a statistical bivariate analysis of the results shown in FIGS. 6A-L for determining if the levels of the biomarker Lp-PLA2 analyzed in different ways vary based on the patient's body mass index (BMI).

FIGS. 11A-C show a statistical one-way analysis of variance (ANOVA) of the results shown in FIGS. 6A-C for determining if the levels of the biomarker Lp-PLA₂ analyzed in different ways vary based on whether the patient was a smoker or non-smoker.

FIG. 12 shows a statistical multivariate correlation analysis of the results shown in FIGS. 6A-C showing if levels of various candidate cardiovascular disease biomarkers including Lp-PLA₂ mass, Lp-PLA₂ total mass (total mass assay) and Lp-PLA₂ activity correlate with each other.

FIGS. 13A-C shows a statistical one-way analysis of variance (ANOVA) of the results shown in FIGS. 6A-C for determining the statistical significance of the levels of the candidate biomarker Lp-PLA₂ analyzed in different ways for patients compared with apparently healthy donors without cardiovascular disease.

FIG. 14A shows a statistical mean and least squares analysis at 95% confidence quartile and ROC curve assay from Lp-PLA₂ standard mass, Lp-PLA₂ total mass and Lp-PLA₂ activity and correlated based on three different assay formats for testing the candidate activity biomarker testing in cardiovascular disease patients compared with apparently healthy individuals.

FIG. 14B shows a statistical logistic regression analysis including AUC of Lp-PLA₂ standard mass and activity biomarkers testing.

FIG. 14C shows a receiver operating characteristic (ROC) curve graphical representation of the data for the Lp-PLA2 mass and activity biomarkers shown in FIG. 19B.

FIG. 14D shows a statistical mean and least squares analysis of breakdown of data for the Lp-PLA₂ standard mass, total mass, and activity biomarkers for different categories of cardiovascular disease.

FIG. 15 show a statistical analysis including cutoffs, 95% confidence intervals, and sensitivity and specificity breakdowns of the results from FIGS. 6A-C showing the improvement in diagnosing cardiovascular disease using normalized levels of biomarkers.

FIG. 16A shows the level of Lp-PLA₂ mass using a standard assay compared with the level of Lp-PLA₂ activity assayed from different fractions of a blood sample that were separated using a sizing column. LDL is found in the first (leftmost peak) while HDL is found in the second (right) peak.

FIG. 16B shows the correlation between the levels of Lp-PLA₂ mass using a total mass assay with the level Lp-PLA₂ activity assayed from various fractions of a blood sample that was separated using a sizing column. Note that the scale for the standard mass assay in FIG. 16A is different from the scale for the total mass assay in FIG. 16B.

FIG. 17 shows a comparison of the effect of the presence of detergent in a human serum blood sample on when Lp-PLA₂ elutes (e.g. with which fraction it elutes) when the sample is separated into samples having different size particles using a sizing column. Note that the graph shows two different scales; the sample that was fractionated without added detergent reads on the left-hand Y axis (which shows a range from 0-60 ng/ml Lp-PLA₂) and the sample that was fractionated in the presence of detergent reads on right-hand Y axis (which shows a range from 0-600 ng/ml). The graph also indicates which fractions contain LDL and HDL.

FIG. 18 shows the effects of the addition of LDL and HDL on Lp-PLA₂ enzyme activity level.

FIG. 19 shows a model of possible interactions between Lp-PLA₂ and LDL.

FIG. 20A shows the degree of correlation between Lp-PLA₂ detected from a blood sample with blood sample fractions that contain chylomicron/VLDL particles, LDL particles, or HDL particles; all assayed using the standard mass assay. The blood sample was separated into the fractions using a sizing column to separate the particles into different fractions based on their different sizes.

FIG. 20B shows the degree of correlation between Lp-PLA₂ detected from a blood sample with blood sample fractions that contain chylomicron/VLDL particles, LDL particles, or HDL particles; all assayed using a total mass assay. The blood sample was separated into the fractions using a sizing column to separate the particles into different fractions based on their different sizes.

FIG. 21 graphically depicts the correlation (or lack thereof) between a mass and an activity of Lp-PLA₂ measured from the same patient.

### DETAILED DESCRIPTION

Described herein are diagnostic biomarker indicators useful for identifying and stratifying cardiovascular disease, and especially for identifying and stratifying early and intermediate stages of cardiovascular disease. Also described are prognostic biomarker indicators that may identify future risk of cardiovascular disease or cardiovascular disease related events. Such biomarker indicators may be more accurate than those provided by currently available markers. Such indicators may be used alone or in conjunction with other indicators described herein or in conjunction with other existing or yet-to-be developed biomarkers. The disclosure also provides methods for addressing a cardiovascular disease state based on the identification and stratification of cardiovascular disease, such as treating a cardiovascular disease, including preventing cardiovascular disease progression or ameliorating effects from cardiovascular disease. These methods for addressing cardiovascular disease may be based on, for example, the prognosis or diagnosis of cardiovascular disease based on one or more than one biomarker or on the level of a biomarker normalized to another biomarker.

In some examples, these biomarkers may be associated with fat or cholesterol manufacture, transport, or degradation in the patient's body. The body produces or absorbs from ingested food various molecules such as lipids and cholesterols, and some of these molecules have been implicated in causing or contributing to CV disease. However, even though the pathways by which these molecules are produced or move through the body have been described, highly sensitive assays to diagnose CVD or predict future risk of CVD are not available and many people with CVD or at risk for CVD do not get identified or appropriately treated.

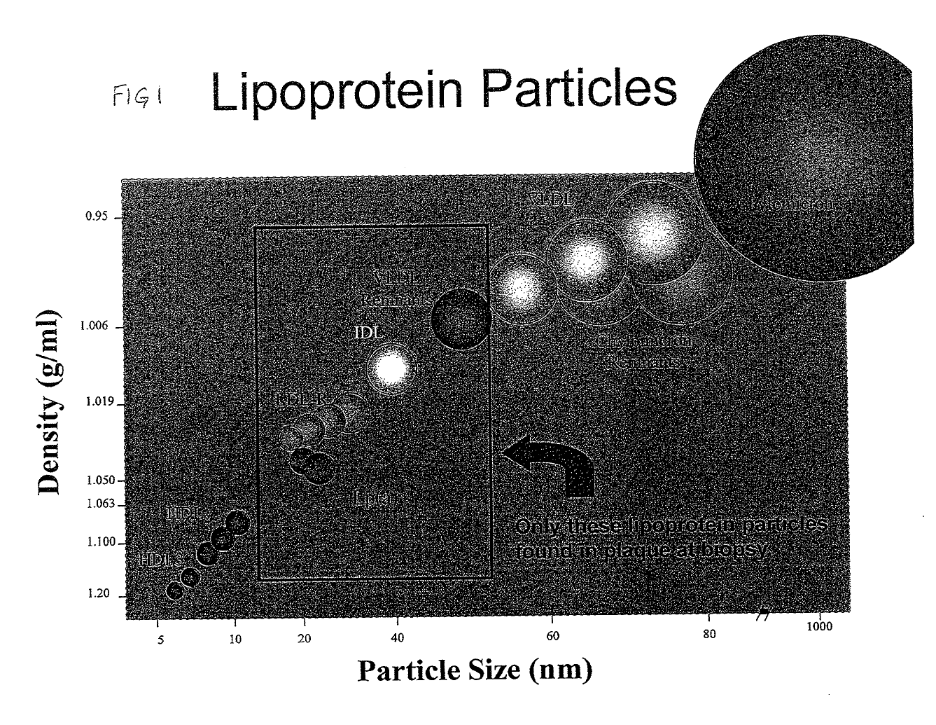

FIG. 1 shows a comparison of different sizes and densities for various types of submicroscopic lipoprotein particles in the body. Lipoprotein particles contain a protein molecule wrapped around cholesterols and fats which is useful for transporting cholesterol and fats through the blood for use by various tissues in the body. FIG. 1 also points to a subset of lipoprotein particles that form part of unwanted plaque deposits on the insides of blood vessels and which are implicated in heart and artery diseases. Both lipoprotein Lp-PLA₂ and cholesterol are associated with lipid vehicles, such as HDL, LDL, chylomicron and VLDL.

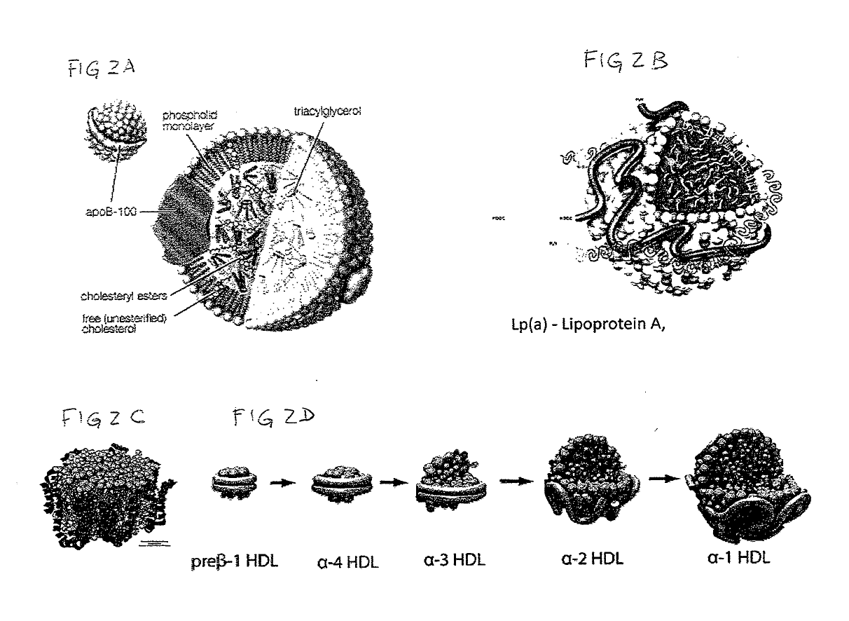

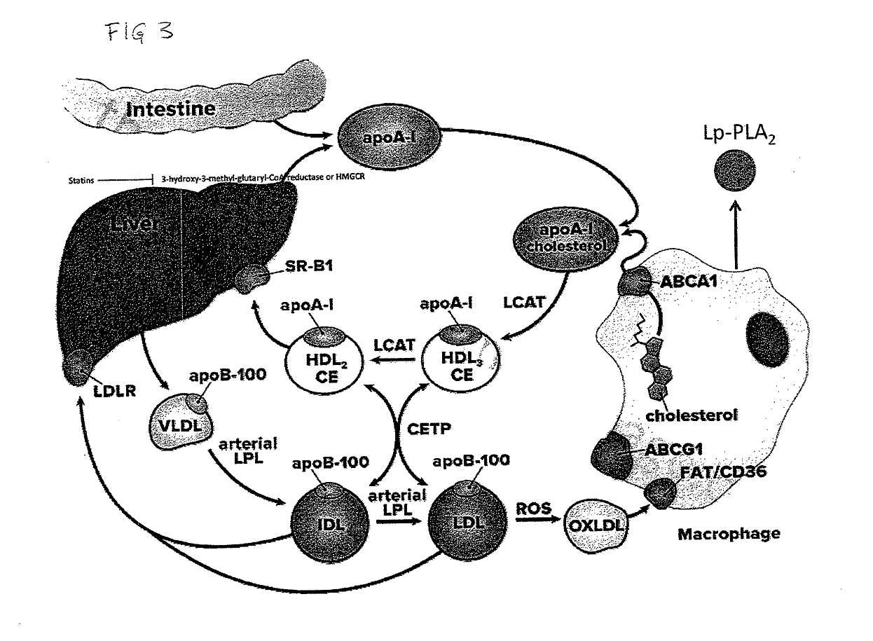

FIG. 2A shows the general structure of one class of lipoproteins shown in FIG. 1, apoB-100 lipoproteins, which have a single particle of an apoliproteinB-100 protein wrapped around an inside of cholesterols and fats and which transport the cholesterol and fats through the blood and into cells.

FIG. 2B shows the general structure of one type of lipoprotein such as shown in FIG. 2A, called lipoprotein (a) (Lp(a)), with a particle of apolipoprotein(a) protein attached to the particle of apoliprotein B-100 protein. High levels of Lp(a) in the bloodstream have been implicated in heart and artery disease.

FIG. 2C shows a space filling model of a mature high-density lipoprotein particle such as one shown in FIG. 1.

FIG. 2D shows the steps of forming a high-density lipoprotein (HDL) particle to form a mature HDL such as one shown in FIG. 2C, by adding additional molecules to the particle to form the mature,

FIG. 3 shows an overview of the molecules involved with lipid and cholesterol synthesis and transport in the blood, and how organs in the body form and change some of these molecules, and in particular how lipoprotein particles form. In particular, FIG. 3 shows molecules that are found in the blood and as such, may be detected using a simple blood test. As mentioned above, both Lp-PLA₂ and cholesterol are associated with lipid vehicles and move through the blood with these vehicles. Cholesterol is found in several forms in the blood: in HDL particles (“good cholesterol”) and in LDL particles (“bad cholesterol”). Atherosclerotic plaques-hard deposits that may impede blood flow or break off and cause a stroke or heart attack-form in the inner lining of an artery and include both Lp-PLA₂ and cholesterol; both are of interest in the development of cardiovascular disease. Both Lp-PLA₂ and cholesterol have been tested and are validated biomarkers tested to indicate cardiovascular disease. The levels of Lp-PLA₂ and cholesterol in the blood can be reduced by statin treatments. Statins are widely used drugs that lower cholesterol levels by inhibiting the action of an enzyme in the liver, 3-hydroxy-3methyl-glutaryl-CoA reductase (HMGCR), as shown in FIG. 3. In previous clinical studies (e.g. Framingham heart study), Lp-PLA₂ activity levels correlated proportionally with LDL-cholesterol (LDL-C) and inversely with HDL-cholesterol. The Framingham Risk Score, which is based on the Framingham heart study, is used to estimate a person's risk of coronary heart disease, and considers the individual's age, gender, total cholesterol, HDL cholesterol, (systolic) blood pressure, and smoking status to determine the individual's risk of coronary heart disease. Some of these factors, such as total cholesterol and systolic blood pressure, have small hazard ratios, typically in the range of 1.5-2.5 (Cook, Clinical Chemistry 54:1 (2008) and it is still difficult to reliably and consistently predict who will have coronary heart disease. A study of a subgroup of placebo controlled men in Scotland (The WOSCOPS trial) for determining the risk factors associated with coronary heart disease concluded that the association of Lp-PLA₂ with coronary heart disease was independent of other, traditionally known heart disease risk factors, such as LDL-cholesterol. Overall, some clinical studies (such as HPS and JUPITER) have shown modest or even negative results using the established biomarker Lp-PLA₂ to prognosticate cardiovascular disease. Additionally, when an individual is treated with a statin, Lp-PLA₂ appears insignificant in the prognosis of cardiovascular disease as shown by the HPS and JUPITER trials (Evan A. Stein, Clinical Chemistry, 58:5, 814-817 2012). Interestingly, the levels of both Lp-PLA₂ and cholesterol decrease in response to statins. There is a need to predict risk and diagnose cardiovascular disease in a patient treated with statins. Thus, there is need for improvement in predicting and diagnosing cardiovascular diseases in general and, more specifically, after a patient undergoes treatment for cardiovascular disease. There is also a need for better understanding and improvement in the use of Lp-PLA₂ as a biomarker of cardiovascular disease. One possible avenue lies in defining the relationship between Lp-PLA₂ and cholesterol.

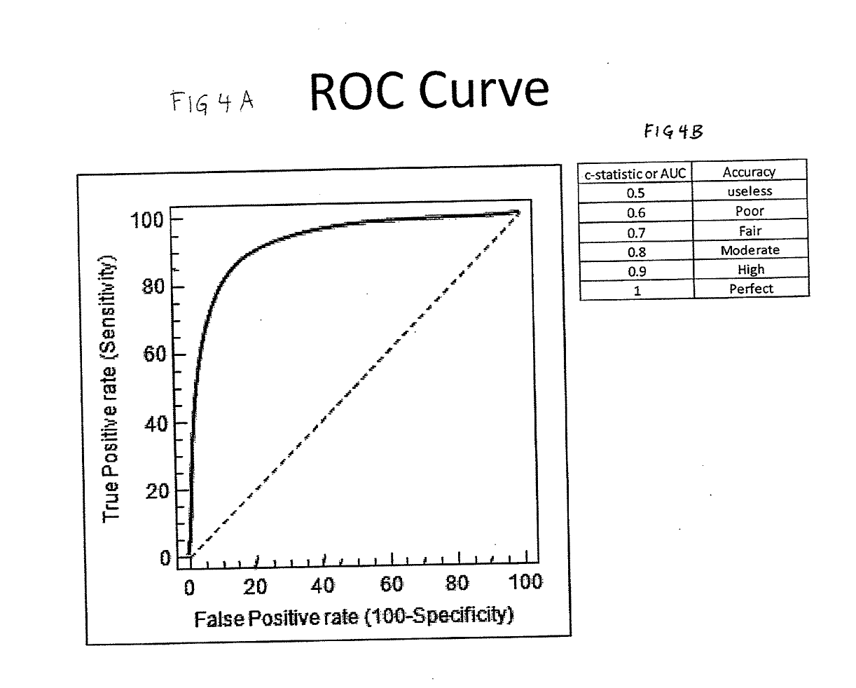

FIG. 4A shows a hypothetical graphical example of a statistically significant result for a biomarker that would be useful for detecting cardiovascular disease using receiver operating characteristic (ROC) analysis. The ROC curve (AUC or c-statistic) shows separation between diseased and non-diseased states. They are widely accepted as the standard method for describing and comparing the accuracy of medical diagnostic tests. ROC curves are independent of the prevalence of disease. Such a curve may be used as a diagnostic model (to identify cardiovascular disease) or as a prognostic model (to predict future risk of cardiovascular disease). Both sensitivity and specificity can be summarized into AUC or c-statistic for evaluation. The ROC curve-shown as the solid line-shows the true positive rate (sensitivity) plotted as a function of the false positive rate (1-specificity). For comparison, a line—the dotted line—is drawn to illustrate random results—a biomarker that shows no correlation to cardiovascular disease. Each point on the ROC curve represents a single sensitivity/specificity pair. A perfect heart failure marker would have a ROC curve that passes through the upper left corner, corresponding to 100% sensitivity and 100% specificity. The closer a biomarker ROC curve is to the upper left hand corner, the better the biomarker is for detecting cardiovascular disease. FIG. 4A also illustrates the area under the ROC curve (AUC), which can vary from 0 to 1 and is useful for determining if use of a particular biomarker is statistically significant for assaying cardiovascular disease. FIG. 4B indicates how an AUC number may be used to determine how useful a biomarker may be for assaying cardiovascular disease, with an AUC biomarker values near 0.5 showing that the biomarker is useless for detecting cardiovascular disease and a biomarker becoming more useful for detecting cardiovascular disease as their AUC values approach 1. The biomarkers-alone and in combination-described herein may be useful for discriminating or classifying cardiovascular disease, including discriminating or classifying subclasses of cardiovascular disease. A ROC curve (e.g. AUC or c-statistic) may be used to determine what biomarkers may be used, what value of a biomarker may be used and what combination of (normalized) biomarkers may be used. A ROC curve (e.g. AUC or c-statistic) may be used to provide a cut-point, a minimum value, or a maximum value. The biomarkers described herein may be useful for reclassifying a disease or a non-disease state. Reclassification is the comparison of the clinical impact between two models. It can be used for evaluating model improvement and clinical impacts. In particular, the biomarkers-alone and in combination-described herein may be useful for diagnosing disease state or reclassifying a previously diagnosed disease state as a non-disease state or reclassifying an individual previously considered non-diseased into a disease state.

Candidate cardiovascular disease biomarkers, including Lp-PLA₂^{Standard mass}, Lp-PLA₂^{Total-mass} and Lp-PLA₂^{Activity}. Lp-PLA₂ (lipoprotein-associated phospholipase A₂) is an enzyme found in the blood that can catalyze the breakdown of oxidative modified polyunsaturated fatty acids into two components, lysophosphatidylcholine (LysoPC) and oxidized nonesterified fatty acids (OxNEFA). It is associated with low-density lipoprotein (LDL) in the blood and its presence correlates with the development of atherosclerosis, coronary heart disease, inflammation, and stroke. It is not known, however, what specific role it might play in the progression or prevention of any of these diseases or if its role might be change under different circumstances. For example, it is not known if Lp-PLA₂ might play a role in causing such diseases or in preventing damage from such diseases. Lp-PLA₂ can be assayed using a standard mass assay (e.g., an Lp-PLA₂^{Standard mass} assay), a total mass assay (e.g., an Lp-PLA₂^{Total mass} assay), or using an activity assay (e.g., Lp-PLA₂^{Activity} assay); these assays measure different qualities of the Lp-PLA₂ molecules and so measure these qualities in different ways. The Lp-PLA₂^{Standard mass} was tested in an ELISA assay using a commercially available kit, PLAC Test ELISA kit, as described below. The Lp-PLA2 total mass was tested in a total mass ELISA assay using a total mass ELISA assay, as described below. The total mass ELISA assay is related to the standard ELISA assay, but includes detergent that allows better detection of Lp-PLA2 mass. The activity assay measures the enzymatic activity of the Lp-PLA₂ enzyme on a substrate.

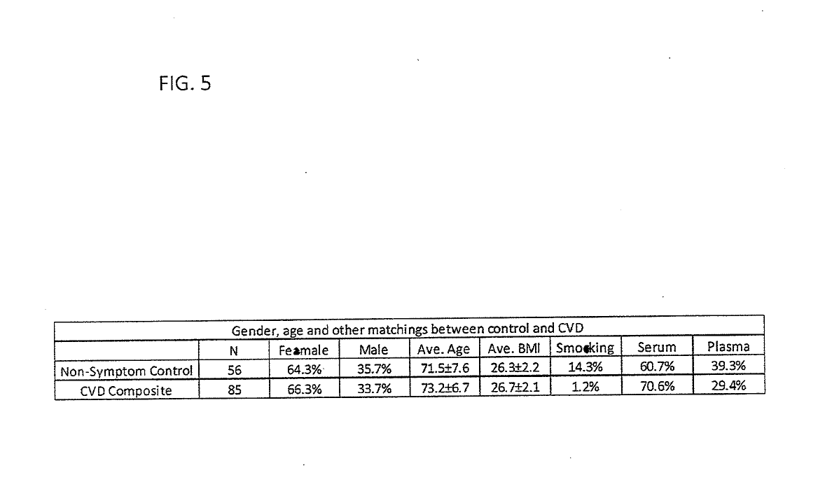

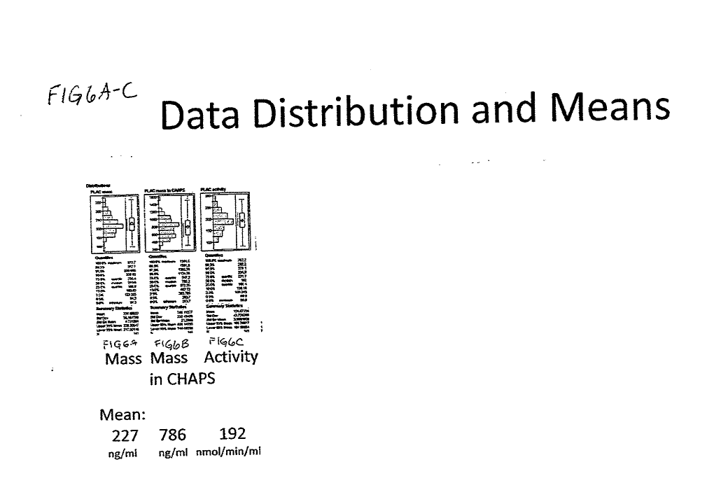

FIG. 5 shows the characteristics of a cohort of patients with cardiovascular disease and a control group population without cardiovascular disease that were tested for biomarkers that may be useful to prognosticate or diagnose cardiovascular disease, as described herein. Levels of the biomarkers Lp-PLA₂ mass and activity, at least, have been reported to vary based on age, community, gender and race. Blood serum or blood plasma samples were collected from CVD individuals and from control Caucasians from Russia in 2011-2013. Samples from both male and female patients were collected. This cohort partially controls for some of these factors that have been proposed to cause the level of, for example, Lp-PLA₂ to vary. The cohort included one hundred and forty one samples comprising eighty-five CVD donor samples (including twenty-nine ischemic and thirteen hemorrhagic stroke, and twenty-five acute myocardial infarction (AMI), seventeen ischemic heart disease (IHD) and fifty-six apparently healthy donor samples. The samples from the CVD cohort were taken when the individuals had experienced their first CVD episode. Stroke diagnosis in the individuals was confirmed by magnetic resonance imaging (MRI) analysis showing a lesion of damaged tissue in the brain of the patient indicative of stroke. The size of the brain lesions ranged from 0.04-0.24 cm². AMI diagnosis was confirmed by both indicative electrocardiogram and elevated troponin-I protein. The troponin-I levels ranged from 2.5-10.5 ug/L. The seventeen IHD patients could not be clearly diagnosed (due to hospital conditions), but all had hypertension. The left ventricle ejection fraction, which might help diagnosis heart failure, was not measured. The age, body mass index (BMI), gender and smoking status of the patients was obtained. The samples were assayed for levels of various components (e.g. biomarkers) and the results subject to standard statistical analyses using a software statistical analysis program (SAS JMP Pro 10.0.2) generally accepted for determining significance of diagnostic assays. In particular, standard Lp-PLA₂ mass was assayed using an Lp-PLA₂^{Standard mass} assay, total Lp-PLA2 was assayed using a total mass assay (e.g., an Lp-PLA₂^{Total mass} assay), and Lp-PLA₂ activity was assayed using an Lp-PLA₂^{Activity} assay). FIGS. 6A-C show median values, mean values, quartiles, range, and standard deviations of the determined levels or the test results from the individuals described in FIG. 5. In particular, values obtained generally show a normal distribution pattern.

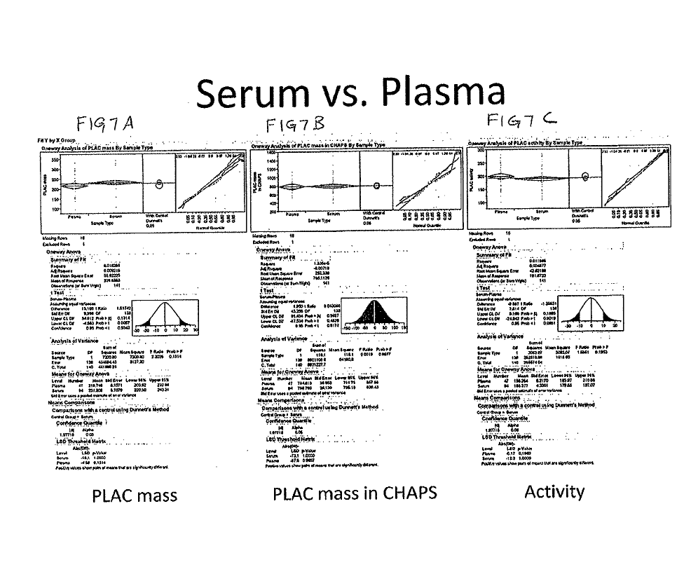

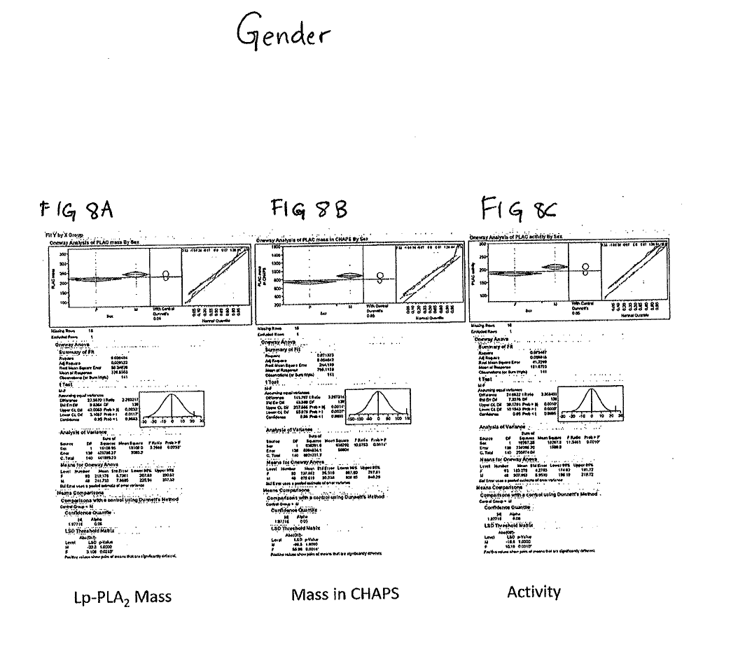

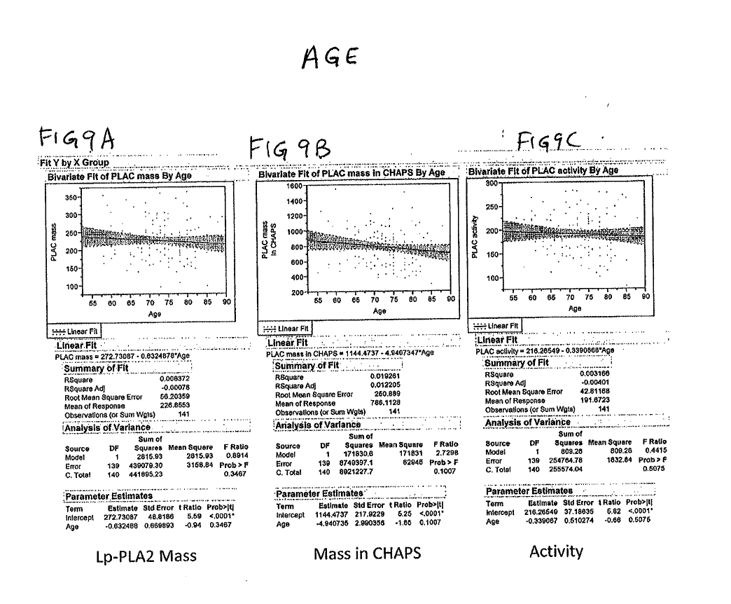

FIGS. 7A-C show a statistical one-way analysis of variance (ANOVA) of the results shown in FIGS. 6A-C, for determining if levels of the candidate biomarkers, including biomarker Lp-PLA₂ which is analyzed by standard mass, total mass and enzyme activity level vary based on the type of blood sample (blood plasma vs blood serum) that was used for testing. The levels of biomarkers assayed by testing serum and plasma were close in value; little or no statistically significant difference was found between samples tested from serum and samples tested from plasma for any of the biomarkers tested. These results are generally consistent with the results reported by Kosaka et al., Clin Chim Acta 2001, October; 312(1-2):179-83, who found no difference between PAF-AH (Lp-PLA₂) activity between blood serum and plasma samples, we also observed no statistically significant differences between the Lp-PLA₂ tested from blood serum and blood plasma samples.

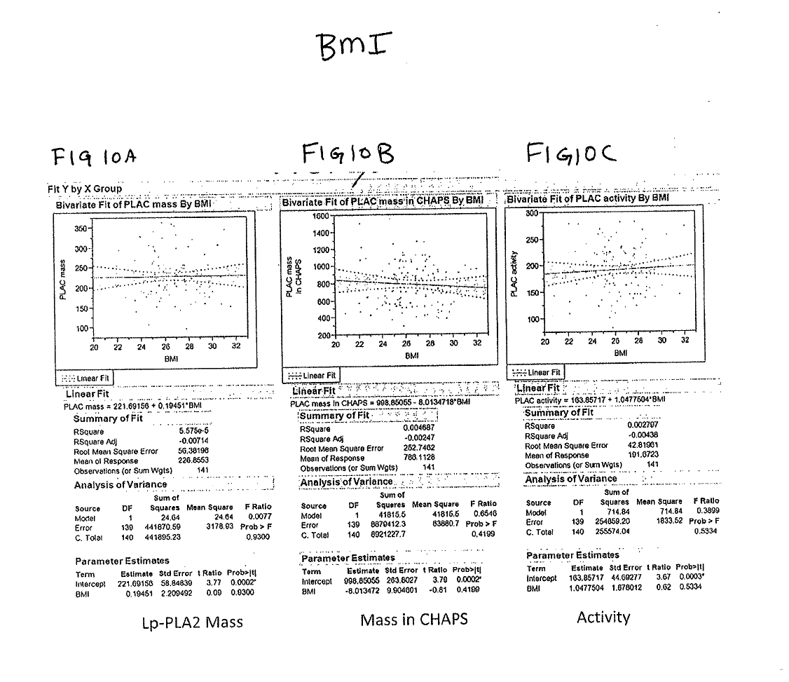

FIGS. 8A-C show a statistical one-way analysis of variance (ANOVA) of the results shown in FIGS. 6A-C, for determining if levels of the candidate biomarkers, including biomarker Lp-PLA₂ which are analyzed by mass level and activity level, vary based on patient gender (whether the patient was male or female). Small but significant gender differences were found between levels of standard Lp-PLA₂ mass, total Lp-PLA2 mass, and Lp-PLA₂ activity. In some examples, gender specific values may be considered for determining a cut-point or threshold for diagnosing cardiovascular disease. In some examples (such as where values are very close to each other), the values may be combined to create a gender-independent cut-point or threshold for diagnosing cardiovascular disease.

FIGS. 9A-C show a statistical bivariate fit analysis of the results shown in FIGS. 6A-C for determining if levels of the candidate biomarkers, including biomarker Lp-PLA₂ which are analyzed in different ways (standard mass, total mass and activity) vary based on patient age. Slight age-specific differences were found between levels of standard Lp-PLA₂ mass, total Lp-PLA₂ mass, and Lp-PLA₂ activity. Yamada et al., Atherosclerosis, 2000, May, 150(1): 209-16, Correlations between plasma platelet-activating factor acetylhydrolase (PAF-AH) activity and PAF-AH genotype, age, and atherosclerosis in a Japanese population showed that plasma PAF-AH activity increased significantly with age in women in the control group with MM and Mm genotypes, and in men in the control group with the MM genotype, but not in men with the Mm genotype. In some examples, age specific values may be considered for determining a cut-point or threshold for diagnosing cardiovascular disease. In some examples (such as where values are very close to each other), the values may be combined to create an age-independent cut-point or threshold for diagnosing cardiovascular disease.

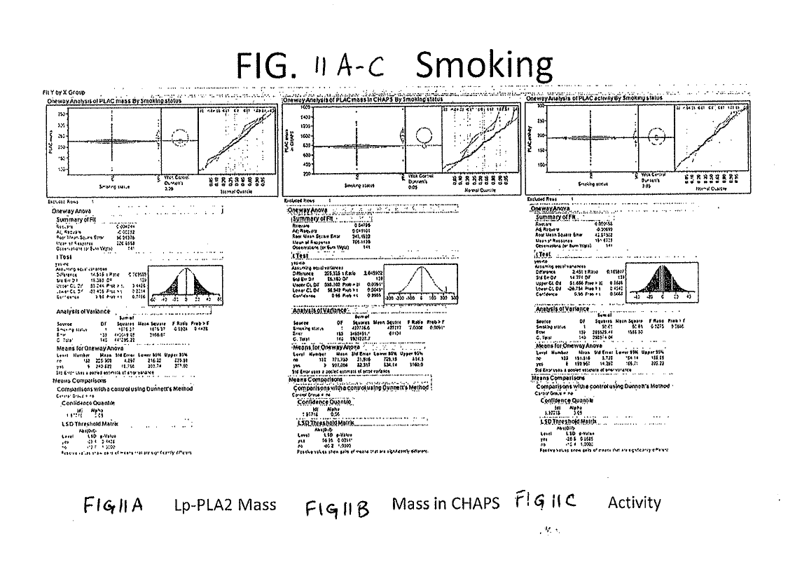

FIGS. 10A-C show a statistical bivariate fit analysis of the results shown in FIGS. 6A-C for determining if levels of the candidate biomarkers, including biomarker Lp-PLA₂ which are analyzed in different ways (standard mass, total mass and activity) vary based on patient body mass index (BMI). Slight differences were found between levels of standard Lp-PLA₂ mass, total Lp-PLA₂ mass, and levels of Lp-PLA₂ activity based on the patient's BMI. In some examples, BMI values may be considered for determining a cut-point or threshold for diagnosing cardiovascular disease. In some examples (such as where values are very close to each other), the values may be combined to create a BMI-independent cut-point or threshold for diagnosing cardiovascular disease.

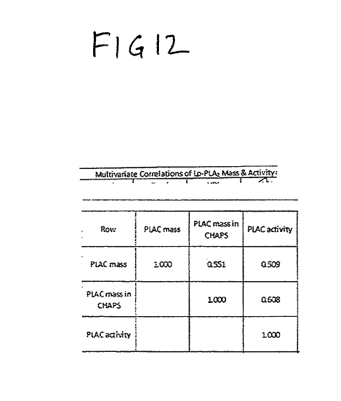

FIGS. 11A-C show a statistical one-way analysis of variance (ANOVA) analysis of the results shown in FIGS. 6A-C for determining if levels of the candidate biomarkers, including biomarker Lp-PLA₂ which are analyzed in different ways (standard mass, total mass and activity) vary based on patient smoking status (whether the patient was a smoker vs non-smoker). However, the number of smokers in the sample was very small and was not statistically significant.

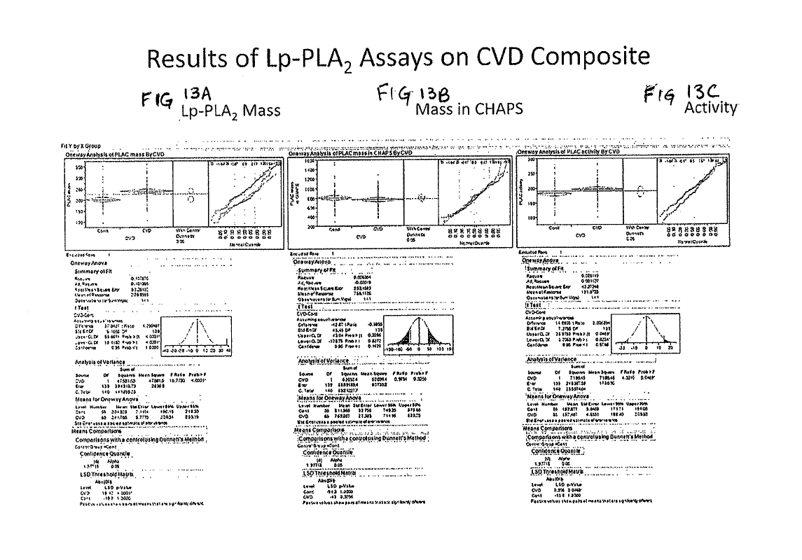

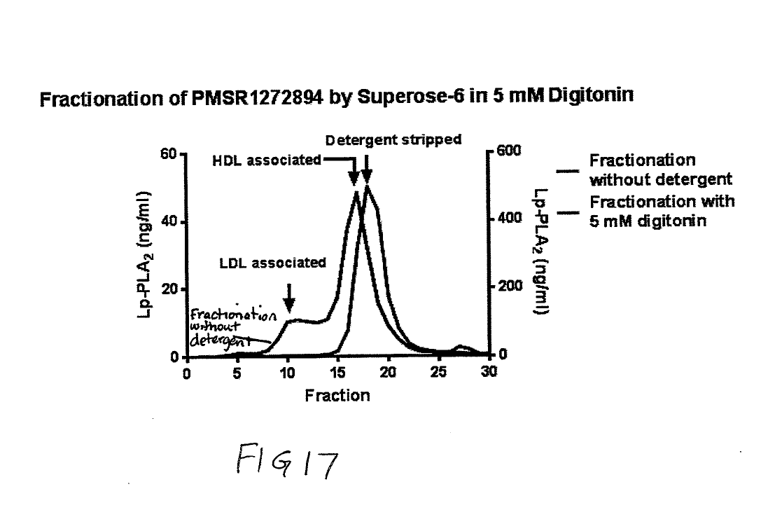

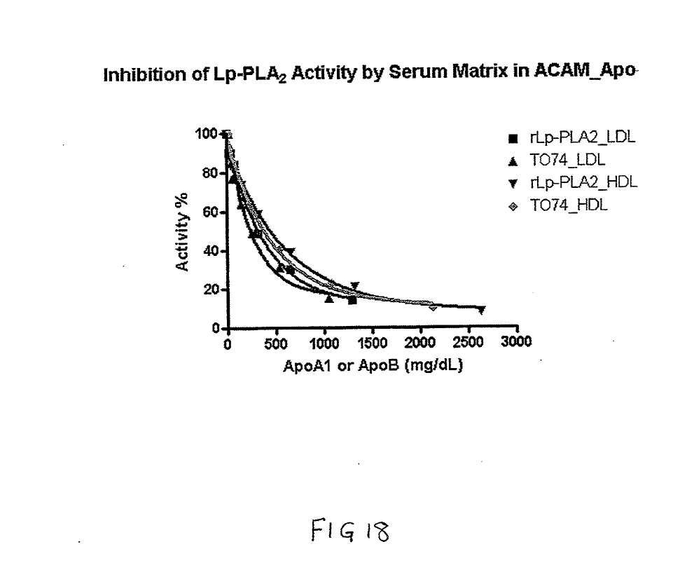

FIG. 12 shows a statistical multivariate correlation analysis of the results shown in FIGS. 6A-C showing the level of correlation between various candidate biomarkers for cardiovascular disease. Correlation (e.g. whether a change in the value of one biomarker predicts a change to the value of a second marker or the degree to which two biomarkers move in tandem) is indicated by a correlation coefficient which may have a value from −1 to 1. A value of zero indicates that there is no correlation between two biomarkers, a value of 1 indicates perfect positive correlation between two biomarkers (e.g. they always move in tandem), and a value of −1 indicates perfect negative correlation between two biomarkers (e.g., they always move in opposite directions). The closer a correlation coefficient is to 1, the greater the positive correlation between two biomarkers; the closer a correlation coefficient is to −1 the greater the opposite or negative correlation between two biomarkers. Generally values between 0 and 0.3 (or −0.3) show weak correlation, values between 0.3 (or −0.3) and 0.7 (or −0.7) show moderate correlation, and values between 0.7 and 1 (or between −0.7 and −1) show strong correlation. FIG. 17 shows results from assaying the levels of standard Lp-PLA₂ mass, total Lp-PLA2 mass, and of Lp-PLA₂ enzyme activity. These assays have aspects that are similar to each other (e.g. all three analyze the same molecule, Lp-PLA₂) and aspects that are different from each other (e.g. the specific details of how the assays are performed to measure standard mass vs total mass vs enzyme activity). Additionally, the activity assay may be affected by the concentration of endogenous phosphotidylcholine, lysophosphotidylcholine, other naturally occurring inhibitors, or gene polymorphisms). These assays give different results in different units (showing a mean value of 227 ng/ml using the Lp-PLA₂^{Mass} assay and a mean value of 192 nmol/min/ml using the Lp-PLA₂^{Activity} assay). Although these assays and results differ, the assays still show moderate correlation with each other (0.509 correlation level). The Lp-PLA₂ activity assay, but shows weak positive association (0.105) with the standard mass assay.

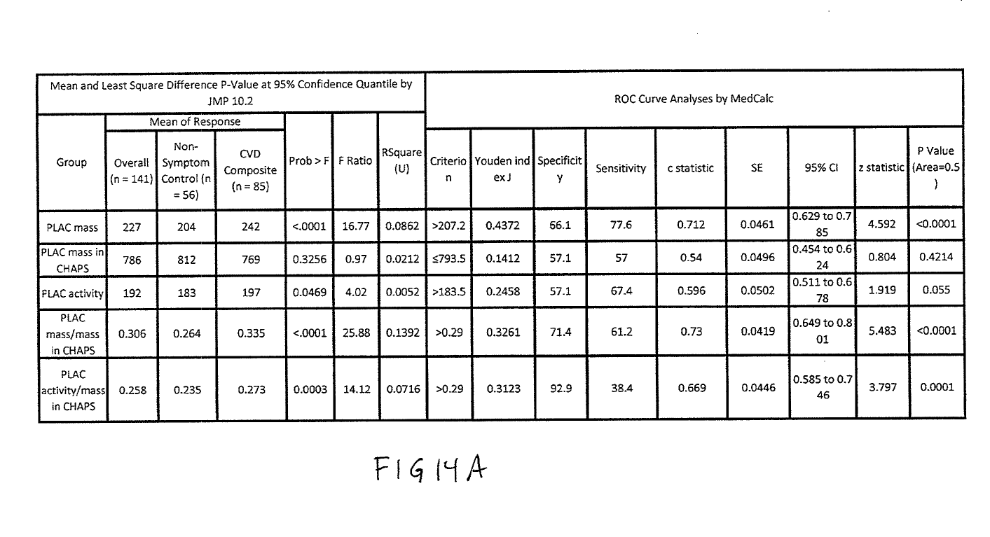

FIGS. 13A-C shows a statistical one-way analysis of variance (ANOVA) of the results shown in FIGS. 6A-C for determining the statistical significance of the levels of the candidate biomarker Lp-PLA₂ analyzed for standard mass, total mass and activity compared with the control group of apparently healthy donors. F is the ratio of two variances. The prob>F is the p value and is a measure of significance (of probability of obtaining a greater F-value by chance alone). A value of 0.05 or less considered evidence of a regression effect. The Lp-PLA₂ mass and activity assays showed significant differences between cardiovascular disease patients and apparently healthy individuals, with F-ratios of 16.7730 and 4.0216, respectively.

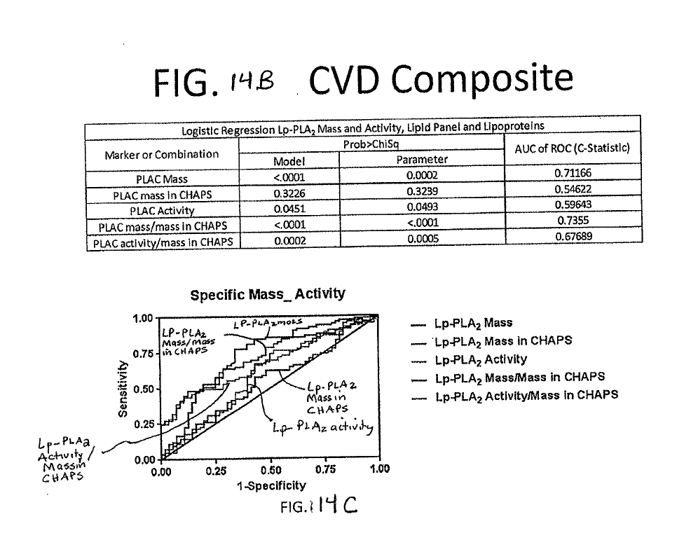

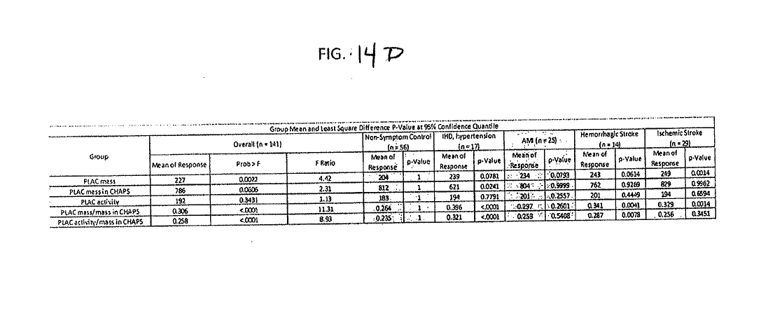

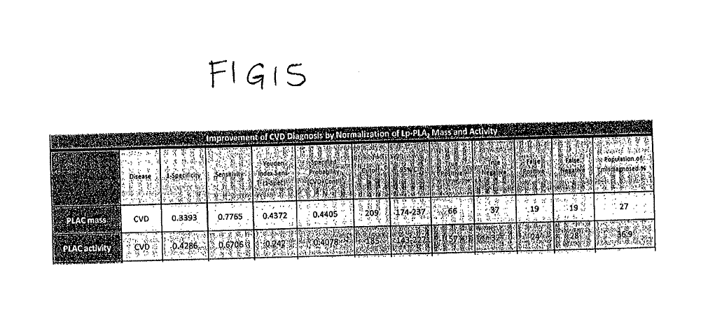

FIG. 14A shows a statistical mean and least squares analysis comparing and correlating results for testing the biomarker Lp-PLA₂ for different characteristics using different assay formats. Any Lp-PLA₂ mass, Lp-PLA₂ total mass or activity values or normalized values for diagnosing cardiovascular disease may be chosen as long as they differentiate individuals into different cardiovascular status categories. For example, a value may be above an overall average test value (e.g. cardiovascular-diseased individuals and non-symptom control values), that are at or above an average value for cardiovascular-diseased individuals, above a non-symptom control value, or may be above a statistically determined value that takes into account both specificity and sensitivity (such as from a Youden index J value from a ROC curve.

FIG. 14A shows that normalizing a level of Lp-PLA2 standard mass to a level of Lp-PLA2 total mass gives a ratio of 0.335 for cardiovascular diseased individuals. This ratio is highly significant for detecting cardiovascular disease, with a statistical F Ratio of 25.88. Compare this value with the F ratio of the standard Lp-PLA₂ mass assay alone, with a lower (but significant) F ratio of 16.77. A level of Lp-PLA₂ standard mass normalized to a level of Lp-PLA₂ total mass may be useful for diagnosing cardiovascular disease and a cut-off or threshold value (as explained above) may be chosen for diagnosing cardiovascular disease state vs non-disease state. For example, a normalized value (e.g. an Lp-PLA₂ standard mass value normalized to an Lp-PLA₂ total mass value) may be at or above 0.306, at or above 0.264, at or above 0.335, or at or above 0.29. Such a normalized value may be used alone, or may be used in conjunction with another value (such as an Lp-PLA₂ standard mass value, Lp-PLA₂ total value, an Lp-PLA₂ activity value or another marker value such as known in the art) for diagnosing cardiovascular disease. A level of Lp-PLA₂ activity normalized to a level of Lp-PLA₂ total mass may be useful for diagnosing cardiovascular disease and a cut-off or threshold value (as explained above) may be chosen for diagnosing cardiovascular disease state vs non-disease state. For example, a normalized value (e.g. an Lp-PLA₂ activity value normalized to an Lp-PLA₂ total mass value) may be at or above 0.258, at or above 0.235, at or above 0.273, or at or above 0.29.

An Lp-PLA₂ standard mass value or Lp-PLA₂ activity value may be used alone or in conjunction with another value, such as an Lp-PLA₂ mass value normalized to an Lp-PLA₂ total mass value. For example, an Lp-PLA₂ standard mass value may be at or above 227 ng/ml, at or above 242 ng/ml, at or above 204 ng/ml, or at or above 207 ng/ml (e.g. 207.2 ng/ml). An Lp-PLA₂ total mass value may be used alone or in conjunction with another value, such as an Lp-PLA₂ standard mass value normalized to an Lp-PLA₂ total mass value. For example, an Lp-PLA₂ total mass value may be at or less than 786 ng/ml, at or less than 812 ng/ml, at or less than 769 ng/ml, or at or less than 794 ng/ml (e.g. 793.5 ng/ml). In practice, changes in the assay format and particulars of test conditions may vary somewhat and therefore different assay runs may give somewhat different absolute values than described here. In some cases, a cut-off value (for determining a healthy or cardiovascular disease status) may be somewhat different. A cut-off value may be calibrated to a control value, and based on normal testing variation, may have an absolute value that is different from that described herein without departing from the scope of the disclosure.

FIG. 14B shows a statistical logistic regression analysis comparing the differences in results (e.g. those shown in FIG. 14A) based on three different assay formats for testing the candidate biomarker Lp-PLA₂.

FIG. 14C shows a receiver operating characteristic (ROC) curve graphical representation of the data shown in FIG. 14B.

FIG. 14D shows a statistical least squares analysis comparing the results from Lp-PLA₂ standard mass levels, Lp-PLA₂ total mass levels, Lp-PLA₂ activity levels, Lp-PLA₂ standard mass levels normalized to Lp-PLA₂ total mass levels, and Lp-PLA₂ activity levels normalized to Lp-PLA₂ total mass levels formats for assaying cardiovascular disease and various cardiovascular disease categories. Normalized values show the greatest significance. Such normalization may, for example, reduce noise in the assay and improve assay performance. A ratio of the levels of Lp-PLA2 assayed/detected using different assays may be useful for identifying cardiovascular disease or for treating a patient for a cardiovascular disease or to prevent initiation or progression of a cardiovascular disease. A ratio useful for identifying cardiovascular disease or risk may be above a certain threshold or may be between a lower threshold and a higher limit. In some cases, an Lp-PLA₂ ratio (and threshold) may be useful for identifying a particular type or risk of a particular type of cardiovascular disease, such as ischemic heart disease or hypertension, acute myocardial infarction, hemorrhagic stroke, or ischemic stroke. For example, a value of Lp-PLA₂ standard mass normalized to a level of Lp-PLA₂ total mass may be above at or 0.306 or at above 0.264 or at or above 0.396 for diagnosing or treating IHD/hypertension. For example, a value of Lp-PLA₂ standard mass normalized to a level of Lp-PLA2 total mass may be above at or 0.306 or at above 0.264 or at or above 0.341 for diagnosing or treating hemorrhagic stroke. For example, a value of Lp-PLA₂ standard mass normalized to a level of Lp-PLA₂ total mass may be above at or 0.306 or at above 0.264 or at or above 0.329 for diagnosing or treating ischemic stroke. For example, a value of Lp-PLA₂ activity normalized to a level of Lp-PLA₂ total mass may be above at or 0.258 or at above 0.235 or at or above 0.321 for diagnosing or treating IHD/hypertension. For example, a value of Lp-PLA2 activity normalized to a level of Lp-PLA₂ total mass may be above at or 00.258 or at above 0.235 or at or above 0.287 for diagnosing or treating hemorrhagic stroke. In some examples, a mass value may be above 227 ng/ml, above 242 ng/ml or above 239 ng/ml for diagnosing hypertension. In some examples, a mass value may above 227 ng/ml, above 242 ng/ml or above 234 ng/ml for diagnosing AMI. In some examples, a mass value may above 227 ng/ml, above 242 ng/ml or above 243 ng/ml for diagnosing hemorrhagic stroke. In some examples, a mass value may above 227 ng/ml, above 242 ng/ml or above 249 ng/ml for diagnosing ischemic stroke. In some examples, an activity value may above 192 nmol/min/ml, above 183 nmol/min/ml, or above 194 nmol/min/ml for diagnosing hypertension. In some examples, an activity value may above 192 nmol/min/ml, above 183 nmol/min/ml, or above 201 nmol/min/ml for diagnosing AMI. In some examples, a mass value may above 192 nmol/min/ml, above 183 nmol/min/ml, or above 201 nmol/min/ml for diagnosing hemorrhagic stroke. In some examples, a mass value may above 192 nmol/min/ml, above 183 nmol/min/ml, or above 194 nmol/min/ml for diagnosing ischemic stroke. Specific tests and test conditions may vary and therefore may give somewhat different absolute values. In some cases, a test value (for determining cardiovascular disease status) may be calibrated to a control value, and based on normal testing variation, may have absolute value that is different from that described herein without departing from the scope of the disclosure. As described above, any of these assays may be used in conjunction with any other assay for diagnosing or treating a cardiovascular disease or cardiovascular disease category. For example, a normalized value of a level of Lp-PLA₂ mass to a level Lp-PLA₂ total mass above 0.306 may be used in conjunction with a minimum value of Lp-PLA₂ mass of 204 ng/ml for diagnosing or treating cardiovascular disease.

FIG. 15 shows a breakdown of the results from FIGS. 6A-C and statistical analyses showing how a level of a biomarker alone or how a ratio of a level of two biomarkers may be useful for diagnosing cardiovascular disease, including specificity and sensitivity calculations. FIG. 15 also show 95% confidence interval for the ratio of the levels of biomarkers described herein, either alone or in combination (e.g. as a ratio). In some examples, a threshold for a cutoff for diagnosing or prognosticating cardiovascular disease or risk may be based on the 95% confidence interval. For example, a threshold may be the lower value, the upper value or a value in between a shown lower value and a shown upper value. F may also be useful for diagnosing (or reclassifying) cardiovascular disease. Such ratios (e.g. normalized values) may be used alone for diagnosing (or reclassifying) cardiovascular disease or may be used together or may be used with other assays. A reclassification may be used to indicate that treating an individual for a cardiovascular disease (including any subcategory or cardiovascular disease) is no longer needed (e.g. that a treatment may be stopped or reduced because they are no longer considered to have a cardiovascular disease). A reclassification may be used to indicate that an individual who previously was not being treated for a cardiovascular disease should be treated. For example, an individual may receive a statin or other treatment.

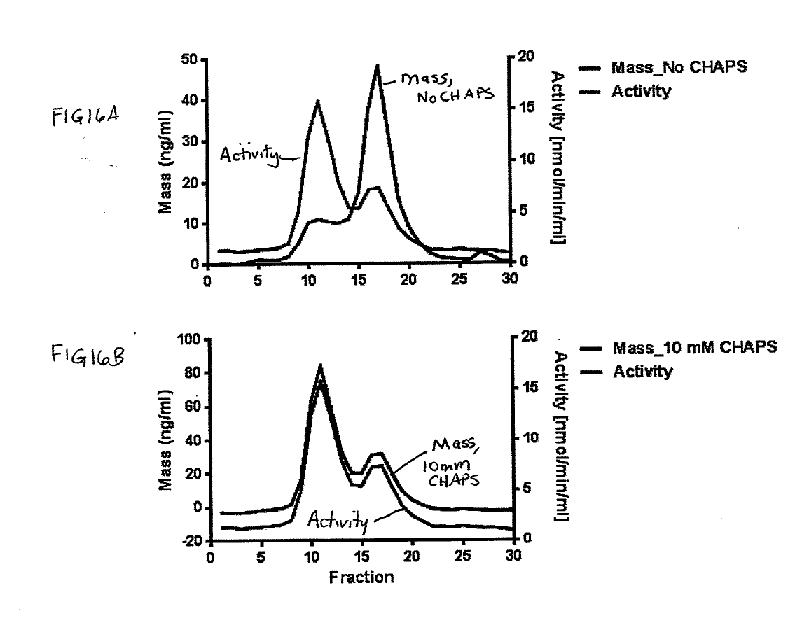

FIG. 16A shows the levels of Lp-PLA₂ mass using a standard assay compared with the levels of Lp-PLA₂ activity as assayed from different fractions of a human blood serum sample that were separated using a Sepharose-6 sizing column. 0.6 ml fractions were collected from the column flowing at 0.3 mls/min and each fraction was used to assay the concentration of Lp-PLA₂ using the standard mass assay and the activity assay. The level of Lp-PLA₂ (scale on the left) assayed using the standard mass assay and the level of Lp-PLA2 assayed using the Lp-PLA₂ activity assay (scale on the right) are shown as a function of the 30 fractions eluted from the column. LDL is found in fractions corresponding to the first (leftmost) peak) while HDL is found in fractions corresponding to the second (right) peak. The assay pattern obtained from the Lp-PLA₂ mass assay is different from the assay pattern obtained from the Lp-PLA₂. In particular, the Lp-PLA₂ mass assay detects Lp-PLA₂ that co-elutes with HDL but detects little Lp-PLA₂ from fractions containing LDL while the Lp-PLA₂ activity assay detects Lp-PLA₂ that co-elutes with LDL but detects less Lp-PLA₂ that co-elutes with HDL.

FIG. 16B shows the correlation between the level of Lp-PLA₂ mass using a total mass assay (as described herein) with the level of Lp-PLA₂ activity assayed from various fractions of the human blood serum sample that was separated using a sizing column as described above for FIG. 16A. The total mass assay is a slightly modified version of the standard mass assay and includes the addition of 10 mM CHAPS to the PBS used for diluting the patient sample and to the conjugate sample. Note that the scale for the Lp-PLA₂ assayed using the standard mass assay in FIG. 16A is different from the scale for the Lp-PLA₂ assayed using the total mass assay in FIG. 16B. The Lp-PLA₂ activity is assayed as described in FIG. 16A; thus the graphs in FIGS. 16A and 16B use the same scale to indicate activity (shown on the right side in both graphs). As can be seen, the pattern of the Lp-PLA₂ mass assay and the pattern of the Lp-PLA₂ activity assay correlate closely with each other. In particular, Lp-PLA₂ mass is now detected in the blood serum fractions that co-elute with LDL (for example in fractions 8, 9, 10, 11, 12, 13, 14 and in some cases fractions 15, 16, and 17) as well as the fractions that co-elute with the HDL (for example in fractions 15, 16, 17, 18, 19, 20, and 21 and in some cases fractions 13 and 14. The addition of CHAPS made the Lp-PLA₂ detectable, presumably by dissociating or otherwise making available for assay the Lp-PLA₂ associated with the Lp-PLA₂. Described herein are assays, materials, and methods useful for assaying Lp-PLA₂ mass, such as total mass including mass that may not be detectable by the standard Lp-PLA₂ assay and which can be made detectable, such as by use of a detergent or the like (e.g. the addition of a detergent or the like to an Lp-PLA₂ assay—the Lp-PLA₂ total mass assay). Such an assay may be useful to detect Lp-PLA₂ as found in one or more of fractions 8, 9, 10, 11, 12, 13, 14, 15, 16, 17, 18, 19, 20, and 21. Such an assay may detect a different amount of Lp-PLA₂ than does a standard mass assay. For example, a total mass assay may detect more than 1×, from 1×-2×, from 2×-4×, from 4×-6×, from 6×-8×, from 8×-10×, or more than 10× the amount of mass compared with a standard Lp-PLA2 mass assay. For example, a total mass assay may detect from 1 ng/ml to 60 ng/ml, from 60 ng/ml to 200 ng/ml, from 200 ng/ml to 400 ng/ml, from 400 ng/ml to 600 ng/ml, from 600 ng/ml to 800 ng/ml or more than 800 ng/ml Lp-PLA₂.

FIG. 17 shows a comparison of the effect of the presence of detergent in a human serum blood sample on when Lp-PLA2 elutes (e.g. with which fraction it elutes) when the sample is separated using a sizing column. Note that the graph shows two different scales which differ by a factor of 10: the sample that was fractionated without added detergent reads on the left-hand Y axis which shows a range from 0-60 ng/ml Lp-PLA₂ while the sample that was fractionated in the presence of detergent reads on right-hand Y axis which shows a range from 0-600 ng/ml Lp-PLA₂. The graph also indicates which fractions contain LDL and HDL. 5 mM digitonin was added in this example to the serum sample prior to fractionation on a Sepharose-6 column as described above; however any detergent (e.g. CHAPS, digitonin, etc.) may be used. The serum sample assayed in the presence of detergent shows a peak (e.g. corresponding to fractions 15, 16, 17, 18, 19, 20, 21, 22) with peak separation from the fractions containing LDL (e.g. fractions 7, 8, 9, 10, 11 and in some cases 12 and 13) and HDL (e.g. fractions 12, 13, 14, 15, 16, 17, 18, 19, 20). The peak in the detergent treated sample may correspond to Lp-PLA2 in a detergent micelle (data not shown). As detergent can readily make Lp-PLA2 from a biological sample such as a blood sample available for assay (e.g. may dissociate Lp-PLA2 from LDL and HDL) some aspects of the invention may use a detergent or the like to for assaying Lp-PLA2 mass. For example, digitonin or any detergent (e.g. BIGCHAP, cephalins, deoxyBIGCHAP, CHAPS, CHAPSO, digitonin, octylglucoside, heptylthioglucoside, phosphatidyl inositol, octylthiglucoside, decylmaltoside, dodecylmaltoside, lecithins, nonylthiomaltoside, MEGA-8, MEGA-9, MEGA-10, potassium sorbate, sodium dodecyl sulfate, sodium propionate, sucrose monocholate, sodium cholate, etc. and hydrates and modifications thereof) may be used for performing an Lp-PLA2 total mass assay. An assay may include a detergent below, at or above a detergent critical micelle formation (CMC), such as a total Lp-PLA₂ standard mass assay. An assay may include formation of a micelle containing a detergent and Lp-PLA₂, such as in a total Lp-PLA2 standard mass assay.

FIG. 18 shows the effects of the addition of either LDL or HDL on Lp-PLA2 enzyme activity levels in an assay. Lp-PLA2 substrate was added either as recombinant protein or as part of a human serum. As described above, apoA1 associates with LDL and the amount of apoA1 was measured as an indication of the amount of LDL. Also as described above, apoB1 associates with HDL and the amount of apoB1 was measured as an indication of the amount of HDL. The squares and up-triangles show the results of the addition of increasing amounts of LDL with either recombinant Lp-PLA₂ protein (rLp-PLA₂; squares) as a control or with human blood serum (TO74; up triangles) containing Lp-PLA₂. In both cases, the Lp-PLA₂ enzyme activity was inhibited by LDL, with increased amounts of LDL showing increased Lp-PLA2 inhibition (decreased activity). The down triangles and diamonds show the results of the addition of increasing amounts of HDL with either recombinant Lp-PLA2 protein (rLp-PLA₂; down triangles) or with human blood serum (TO74; diamonds). In both cases the Lp-PLA₂ enzyme activity was inhibited by the HDL with increased amounts of HDL showing increased Lp-PLA₂ inhibition (decreased activity).

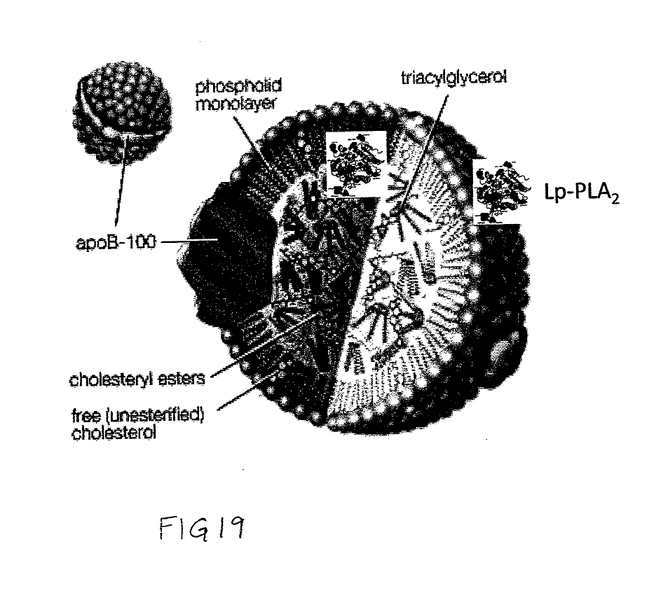

FIG. 19 shows a proposed model of possible interactions between Lp-PLA₂ and LDL. Some of the Lp-PLA₂ is available (shown as Lp-PLA₂ on the surface) and detectable by both the standard Lp-PLA₂ mass assay and the total Lp-PLA₂ mass assay (as well as the activity assay). Some Lp-PLA₂ is unavailable (shown as Lp-PLA₂ on the inside of the particle) and is not detectable by the standard Lp-PLA₂ mass assay, but is detectable by the total Lp-PLA₂ mass assay (and by the Lp-PLA₂ activity). For example, the addition of detergent may make the Lp-PLA2 shown inside the LDL particle available for assay.

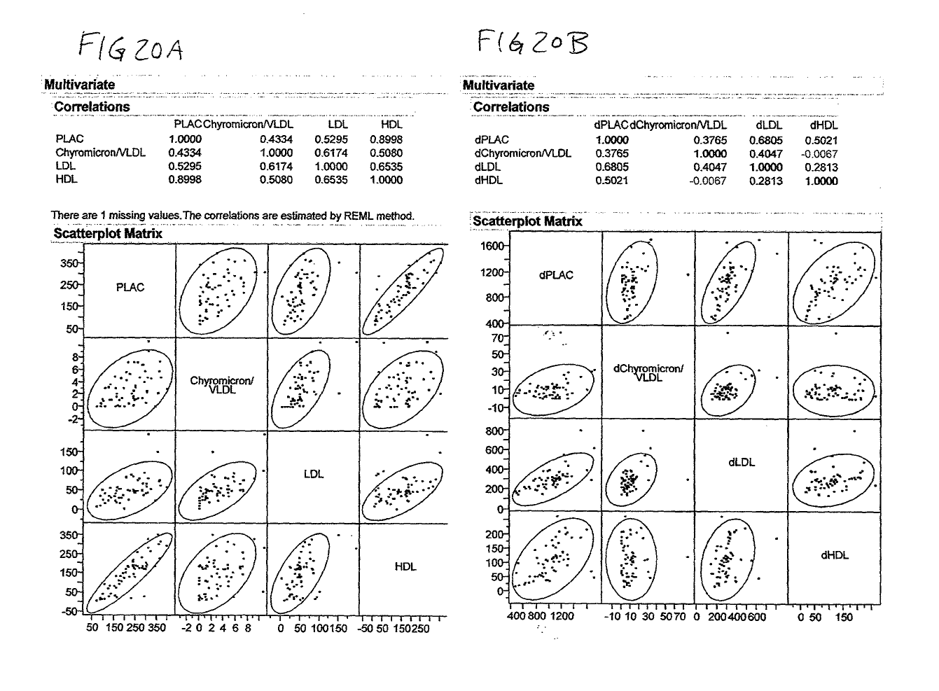

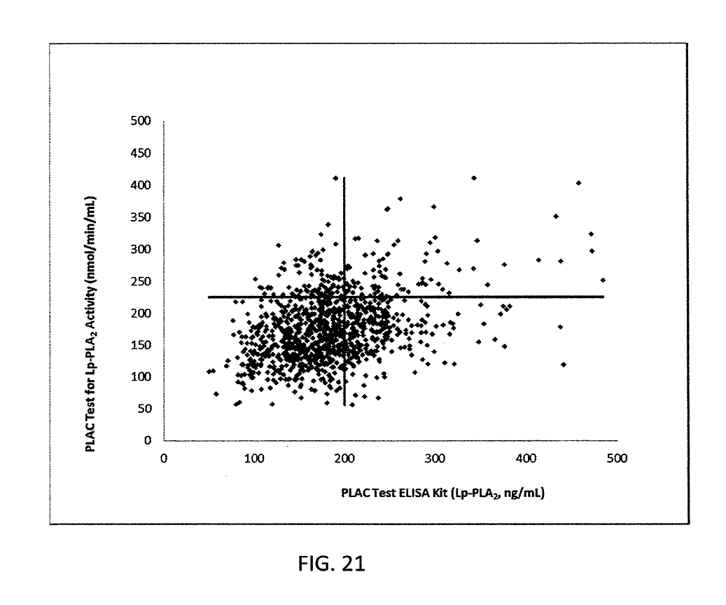

FIG. 20A shows the degree of correlation between Lp-PLA₂ assayed using the standard mass assay and chylomicron/VLDL particles, LDL particles, and HDL particles from fractions from in a blood sample separated using a Sepharose-6 sizing column as described above. Lp-PLA₂ and HDL show the highest correlation, 0.8998, suggesting that the standard Lp-PLA₂ assay may detect Lp-PLA₂ associated with HDL. A lower level of correlation (association) is noted between Lp-PLA₂ and LDL.

FIG. 20B shows the degree of correlation between Lp-PLA2 assayed using the Lp-PLA2 total mass assay (with detergent) and chylomicron/VLDL particles, LDL particles, and HDL particles from fractions from in a blood sample separated using a sizing column. Lp-PLA2 and LDL show the highest correlation, 0.6805 in the presence of detergent suggesting that the total mass assay detects Lp-PLA2 associated with LDL.

A treatment for heart failure may be or may involve any type treatment as known in the art, such as administering a medication, using a medical device, surgery, or using another type of treatment. A treatment may include a administering a medication, such as administering an aldosterone antagonist, an angiotensin-converting enzyme inhibitor, an angiotensin II receptor blocker, a beta blocker, digoxin, a diuretic, an inotrope. A treatment may include a performing a surgery, such as performing a coronary bypass surgery, heart valve repair or replacement, an implantable cardioverter-defibrillator (ICD), cardiac resynchronization therapy, a heart pump, or a heart transplant. Another type of treatment may include, for example, implanting stem cells such as cardiac or other stem cells.

### Treatment Methods

As mentioned above, the techniques described herein may be used to treat or prevent cardiovascular disease. For example, a method of treating or preventing cardiovascular disease (e.g., in a patient previously undiagnosed as having cardiovascular disease) may include detecting a level of Lp-PLA₂ (e.g., mass or activity) either alone or in a ratio (e.g. normalized value) in combination with one or more other biomarkers (e.g., HDL-C, apoA1, etc.) and treating the patient by prescribing a therapy to treat cardiovascular disease based on the level of Lp-PLA₂ alone or to the ratio of Lp-PLA₂ to one or more other biomarker. Any appropriate therapy may be used, but may in particular include a pharmaceutical agent (e.g., composition, compound, drug). Examples of such pharmaceutical agents includes: aldosterone blockers, angiotensin-converting enzyme (ACE) inhibitors, angiotensin-receptor blockers (ARBs), aspirin, beta blockers, diuretics, digitalis, hydralazine and nitrates, statins, and warfarin.

Angiotensin-converting enzyme (ACE) inhibitors are often used for treating patients with heart failure. ACE inhibitors open blood vessels and decrease the workload of the heart. They are used to treat high blood pressure but can also help improve heart and lung muscle function. ACE inhibitors are particularly important for patients with diabetes, because they also help slow progression of kidney disease.

Angiotensin-Receptor Blockers (ARBs), also known as angiotensin II receptor antagonists, are similar to ACE inhibitors in their ability to open blood vessels and lower blood pressure. They may have fewer or less-severe side effects than ACE inhibitors, especially coughing, and are sometimes prescribed as an alternative to ACE inhibitors. Some patients with heart failure take an ACE inhibitor along with an ARB.

Beta blockers are almost always used in combination with other drugs, such as ACE inhibitors and diuretics. They help slow heart rate and lower blood pressure. When used properly, beta blockers can reduce the risk of death or re-hospitalization. Beta blockers can lower HDL (“good”) cholesterol, so have not previously been used with patients having a high level of Lp-PLA2.

Diuretics cause the kidneys to rid the body of excess salt and water. Fluid retention is a major symptom of heart failure. Aggressive use of diuretics can help eliminate excess body fluids, while reducing hospitalizations and improving exercise capacity. These drugs are also important to help prevent heart failure in patients with high blood pressure. In addition, certain diuretics, notably spironolactone (Aldactone), block aldosterone, a hormone involved in heart failure. This drug class is beneficial for patients with more severe heart failure (Stages C and D). Patients taking diuretics usually take a daily dose. Diuretics, or any of the treatments described herein, may be modified based on the level of Lp-PLA₂ or Lp-PLA₂ in combination with one or more other biomarkers. For example, the amount and timing of the diuretic (or other heart failure agent) may be adjusted on this basis.

Aldosterone is a hormone that is critical in controlling the body's balance of salt and water. Excessive levels may play important roles in hypertension and heart failure. Drugs that block aldosterone are prescribed for some patients with symptomatic heart failure. They have been found to reduce mortality or death rates for patients with heart failure and coronary artery disease, especially after a heart attack. These blockers pose some risk for high potassium levels.

Digitalis is derived from the foxglove plant. It has been used to treat heart disease since the 1700s. Digoxin (Lanoxin) is the most commonly prescribed digitalis preparation. Digoxin decreases heart size and reduces certain heart rhythm disturbances (arrhythmias). Unfortunately, digitalis does not reduce mortality rates, although it does reduce hospitalizations and worsening of heart failure. Controversy has been ongoing for more than 100 years over whether the benefits of digitalis outweigh its risks and adverse effects. Digitalis may be useful for select patients with left-ventricular systolic dysfunction who do not respond to other drugs (diuretics, ACE inhibitors). It may also be used for patients who have atrial fibrillation.

Hydralazine and nitrates are two older drugs that help relax arteries and veins, thereby reducing the heart's workload and allowing more blood to reach the tissues. They are used primarily for patients who are unable to tolerate ACE inhibitors and angiotensin receptor blockers. In 2005, the FDA approved BiDil, a drug that combines isosorbide dinitrate and hydralazine. BiDil is approved to specifically treat heart failure in African-American patients.

Statins are important drugs used to lower cholesterol and to prevent heart disease that can lead to heart failure. These drugs include lovastatin (Mevacor), pravastatin (Pravachol), simvastatin (Zocor), fluvastatin (Lescol), atorvastatin (Lipitor), and rosuvastatin (Crestor). In 2007, the Food and Drug Administration (FDA) approved atorvastatin to reduce the risks for hospitalization for heart failure in patients with heart disease.

Aspirin is a type of non-steroid anti-inflammatory (NSAID). Aspirin is recommended for preventing death in patients with heart disease, and can safely be used with ACE inhibitors, particularly when it is taken in lower dosages (75-81 mg).

In particular, the techniques described herein may be used to treat a subject by providing aspirin (e.g., acetylsalicylic acid) when the subject's level of Lp-PLA₂ exceeds a threshold (e.g., >about 400 ng/ml) alone or in combination with one or more other biomarkers. Curiously, previous work has taught away from the use of aspirin when the level of Lp-PLA₂ is above normal in a patient. See, e.g., Hatoum et al. “Dietary, lifestyle, and clinical predictors of lipoprotein-associated phospholipase A2 activity in individuals without coronary artery disease” in Am J Clin Nutr 2010; 91:786-93. (“Aspirin use was also positively associated with Lp-PLA₂ activity”).

Warfarin (Coumadin) is generally recommended only for patients with heart failure who also have: atrial fibrillation, a history of blood clots to the lungs, stroke, or transient ischemic attack, a blood clot in one of their heart chambers. Other drugs that may be used may include Nesiritide (Natrecor), Erythropoietin, Tolvaptan, Levosimendan, etc.

### EXAMPLES

### Example 1

The levels of Lp-PLA₂ mass in plasma and serum samples from the cohort of patients with cardiovascular disease and a control group population without cardiovascular disease were tested for using a commercially available Lp-PLA₂ Enzyme-linked sandwich immunosorbent assay (ELISA) (Gen-3; diaDexus, Inc., South San Francisco, Calif.). The ELISA kit uses two highly specific monoclonal antibodies for measurement of Lp-PLA₂ concentration. The microwell plate is coated with mouse monoclonal anti-Lp-PLA₂ (2C10) antibody.

### Preparatory Steps

1. Bring the microwell plate, Conjugate, Wash Buffer and TMB to room temperature (20 to 26° C.) before use.

2. Remove the microwell plate frame and the required number of coated microwell strips from the foil pouch. Completely reseal the foil pouch containing any unused strips with the desiccant that came in the pouch and store at 2 to 8° C.

3. Prepare 1× Wash Buffer by diluting 20× Wash Buffer 1:20 with deionized water (1 part Wash Buffer and 19 parts of deionized water). Store at room temperature (20 to 26° C.). Use 1× Wash Buffer within four weeks of preparation.

4. Allow patient samples to thaw at 2 to 8° C., if needed, and place on ice or at 2 to 8° C. as soon as thawed.

5. Store the Controls at 2 to 8° C. or on ice until used.

6. Vortex the samples and Controls to mix thoroughly. Avoid foaming.

### Sample Incubation

1. Using a pipettor and tip with appropriate low volume precision, dispense 20 μL of Calibrators, samples and Controls into the appropriate wells after vortexing. Use a calibrated pipette and new pipette tip for each Calibrator, Control or sample.

2. Allow the samples to incubate on the microwell plate for 10±2 minutes before adding the Conjugate.

3. Pipette 200 μL of room temperature Conjugate into the appropriate wells of the coated microwell plate. Avoid contamination by adding the Conjugate without touching the samples with the pipette tips. If there is cross over, change tips and continue adding Conjugate to the wells.

4. Incubate for 3 hours at room temperature.

5. At the end of the incubation period, wash the microwells four (4) times with at least 300 μL of the supplied room temperature 1× Wash Buffer. (DO NOT USE TAP or DISTILLED WATER.)

6. Blot the microwell plate on absorbent paper after the final wash. Immediately (in less than 2 minutes) proceed to the next step. Do not allow the microwell plate to dry.

### Substrate Incubation

1. Pipette 100 μL of room temperature TMB Reagent into each well.

2. Gently swirl the microwell plate on a flat surface for 10 to 15 seconds to ensure mixing.

3. Incubate the microwell plate at room temperature for 20 minutes in the dark.

4. Stop the reaction by adding 100 μL of room temperature Stop Solution to each well.

5. Gently swirl the microwell plate on a flat surface for 20 to 30 seconds to ensure mixing. It is important to make sure that the blue color completely changes to yellow color.

6. Wipe moisture from the bottom of the microwell plate using a paper towel.

7. Within 15 minutes of adding the Stop Solution, read the optical density (O.D.) at 450 nm using a microwell plate reader.

### Example 2

The levels of Lp-PLA₂ mass in plasma and serum samples from the cohort of patients with cardiovascular disease and a control group population without cardiovascular disease were tested for using a commercially available Lp-PLA₂ Activity assay (PLAC® Test for Lp-PLA₂ activity (diaDexus, Inc., South San Francisco, Calif.) for the quantitative determination of Lp-PLA2 activity in human plasma and serum on an automated clinical chemistry analyzer.

The PLAC Test for Lp-PLA₂ Activity has been run using the Beckman Coulter (Olympus) AU400® Analyzer.

Settings for the Beckman Coulter (Olympus) AU400® Clinical Analyzer

Assay Code Rate

Assay Time 8.5 minutes

Read Cycle 12 to 14

Sample Volume 25 μL

Reagent R1 vol. 100 μL R1 reagent (R1 position)

Reagent R2 vol. 25 μL R2 reagent (R2 position)

Wavelength 1° 410 nm, 2° 520 nm

Calibration Method Spline 5 point

Assay Range 1.4 to 400 nmol/min/mL

All samples have been well mixed before testing.

R1: 0.2 M HEPES, pH 7.60, and 10 mM Sodium nonanesulfonate (SNS)

R2: 20 mM citric acid, pH 4.5, containing 10 mM SNS and 0.95-1% 1-myristoyl-2-(4-nitrophenylsuccinyl) phosphatidylcholine (final concentration: 0.15 mM).

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of diagnosing or assessing cardiovascular disease (CVD) in a patient, the method comprising:
detecting a first level of a first assayable Lp-PLA₂ from the patient;
detecting a second level of a second assayable Lp-PLA2 from the patient which second level is different from the first level;
determining a value of the first level of Lp-PLA₂ normalized by the level of the second level to generate a normalized level of Lp-PLA₂; and
diagnosing or assessing cardiovascular disease based on the normalized level.

2. The method of claim 1 wherein detecting a first level comprises detecting an Lp-PLA₂ mass level.

3. The method of claim 1 wherein detecting a first level comprises detecting an Lp-PLA₂ activity level.

4. The method of claim 1 wherein detecting a first level of a first assayable Lp-PLA₂ comprises detecting an Lp-PLA2 mass in the absence of a detergent.

5. The method of claim 1 wherein the second assayable Lp-PLA₂ comprises Lp-PLA₂ assayable in the presence of a detergent that is not assayable in the absence of the detergent.

6. The method of claim 1 wherein the first level comprises a first Lp-PLA₂ mass level and the second level comprises an Lp-PLA₂ activity level.

7. The method of claim 1 wherein the first level comprises a first Lp-PLA₂ mass level and the second level comprises a second Lp-PLA₂ mass level.

8. The method of claim 1 wherein the first level comprises a first Lp-PLA₂ activity level and the second level comprises a second Lp-PLA₂ mass level.

9. The method of claim 1 wherein diagnosing or assessing further comprises determining a minimum level of the first assayable Lp-PLA₂ or the second assayable Lp-PLA₂.

10. The method of claim 13 wherein determining the minimum level of the first assayable Lp-PLA₂ comprises determining an Lp-PLA₂ mass level at least about 207 ng/ml.

11. The method of claim 10 wherein determining the minimum level of the first assayable Lp-PLA₂ comprises determining an Lp-PLA₂ enzyme activity level at least about 184 nmol/min/ml.

12. The method of claim 1 wherein diagnosing or assessing comprises diagnosing or assessing heart disease, acute myocardial infarction, or stroke.

13. The method of claim 1 further comprising providing therapy to the patient when the normalized level is above a first threshold, wherein the therapy for cardiovascular disease is a pharmaceutical agent.

14. The method of claim 1 further comprising providing therapy to the patient when the value is above a first threshold, wherein the therapy for cardiovascular disease is selected from the group consisting of: aldosterone blockers, angiotensin-converting enzyme (ACE) inhibitors, angiotensin-receptor blockers (ARBs), aspirin, beta blockers, diuretics, digitalis, hydralazine and nitrates, statins, and warfarin.

15. A non-transitory computer-readable storage medium storing a set of instructions capable of being executed by a processor, that when executed by the processor causes the processor to:
receive a patient's first level of a first assayable Lp-PLA₂;
receive the patient's second level of a second assayable Lp-PLA₂;
determine a normalized level of Lp-PLA₂ by normalizing the received level of the first assayable Lp-PLA₂ by the received level of the second assayable Lp-PLA₂; and
output the normalized level of Lp-PLA₂ specific to the patient.

16. The non-transitory computer-readable storage medium of claim 15 wherein the set of instructions, when executed by the processor, further causes the processor to indicate if the normalized level of Lp-PLA₂ is above a threshold value.

17. The non-transitory computer-readable storage medium of claim 15 wherein the set of instructions, when executed by the processor, further causes the processor to provide a treatment recommendation for the patient based on the normalized level of Lp-PLA₂.

18. The non-transitory computer-readable storage medium of claim 15, wherein the processor comprises a microprocessor.

19. The non-transitory computer-readable storage medium of claim 15, wherein the processor comprises a smartphone.

20. An Lp-PLA₂ assay for determining Lp-PLA₂ mass comprising:
a buffer solution comprising a detergent;
a substrate comprising an anti-Lp-PLA₂ antibody that recognizes Lp-PLA₂ protein; and
a colorometric or fluorescent reagent configured to produce a detectable signal after Lp-PLA₂ contacts the antibody.

21. The assay of claim 20 wherein the detergent comprises an amount at or above a level for detergent critical micelle formation.

22. The assay of claim 20 wherein the detergent comprises CHAPS.

23. The assay of claim 20 comprising a second antibody that recognizes the anti-Lp-PLA₂ antibody.

24. The assay of claim 20 further comprising an immobilized peroxidase on the anti-Lp-PLA₂ antibody.

25. The assay of claim 20 wherein the immobilize peroxidase comprises horseradish peroxidase.

26. The assay of claim 20 wherein the reagent is a colorometric reagent comprising 3, 3′,5,5′-tetrametylbenzidine (TMB).

27. The assay of claim 20 wherein the substrate comprises a tube or a microwell plate.

28. The assay of claim 20 wherein the detectable signal is detectable using light at around 450 nm.
